(19) United States
(12) Patent Application Publication
(10) Pub. No.: US 2020/0268995 A1
(43) Pub. Date: Aug. 27, 2020

(54) AIR PURIFYING MASK CAPABLE OF FILTERING INHALED AND EXHALED AIR

(71) Applicant: Dong Jin SHIN, Seoul (KR)

(72) Inventor: Dong Jin SHIN, Seoul (KR)

(21) Appl. No.: 16/079,077

(22) PCT Filed: Dec. 7, 2017
(86) PCT No.: PCT/KR2017/014301

§ 371 Date: Aug. 22, 2018

(30) Foreign Application Priority Data
Mar. 8, 2017 (KR) 10-2017-0029491

(51) Int. Cl. A61M 16/06; A61M 16/10

(52) U.S. Cl. CPC A61M 16/06; A61M 16/1065; A61M 2202/203; A61M 2202/206; A61M 16/107

(57) ABSTRACT

The present invention relates to an air purifying mask capable of filtering inhaled and exhaled air that filters and purifies, at the time when external air is absorbed to the interior thereof and even at the time when the exhaled air is exhausted therefrom, a variety of bacteria and fine foreign substances contained in the exhaled air. According to the present invention, the air purifying mask includes: an inhaled air opening part open at the time of inhalation and closed at the time of exhalation in such a manner as to be coupled to an intake air filtering part for filtering and introducing external air; and an exhaled air opening part open at the time of the exhalation and closed at the time of the inhalation in such a manner as to be coupled to an exhaust air filtering part for filtering and exhausting the exhaled air.

### BACKGROUND OF THE INVENTION

### Field of the Invention

The present invention relates to an air purifying mask having an exhaust air purification function, and more particularly, to an air purifying mask that is capable of filtering and purifying, at the time when external air is absorbed to the interior thereof and even at the time when exhaled air is exhausted therefrom, a variety of bacteria and fine foreign substances contained in the exhaled air, thereby in advance preventing the infection of the respiratory diseases caused by the respiration of a carrier or suspected patient.

In detail, the present invention relates to an air purifying mask capable of filtering inhaled and exhaled air that is configured to allow an intake part through which external air is introduced into the mask to be open at the time of air intake and to be closed at the time of air exhaust and also to completely prevent occurrence of a given gap on the intake part at the time of the air exhaust, thereby keeping the exhaled air of a carrier or suspected patient from leaking to the intake part.

### Background of the Related Art

Generally, the easiest method of blocking foreign substances, dust, bacteria or virus contained in the air is to wear a mask.

Since a lot of people are exposed to yellow sand when the seasons changed, that is, in spring or fall season, especially, a variety of respiratory diseases like allergy, asthma and so on may be generated, and further, as recently prevailed novel influenza (H1N1) and middle east respiratory syndrome (MERS) are infected through the nose and mouth, the importance of the mask becomes more seriously emerged.

In conventional practices, a general mask includes a body for covering a wearer's nose and mouth and wearing bands for locking the body onto the wearer's ears.

The body of the conventional mask is made simply by cutting and backstitching a general cotton or fiber material, so that there is a limitation in blocking fine dust, toxic gas, bacteria or virus contained in the air.

So as to remove such problems, a dust or gas mask using a metal or synthetic resin filter to provide a good filtration and purification function has been proposed, but in this case, the dust or gas mask just filters and purifies external air, so that it does not purify the exhaled air of the wearer.

In case of the middle east respiratory syndrome (MERS) recently prevailed in Korea, persons who take care of confirmed cases or suspected cases and even persons who stay for a while in the same place as the patients or suspected cases stay may be infected only with the exhaled air of the confirmed cases or suspected cases, and so as to prevent such infection, accordingly, there is a definite need for a novel mask capable of filtering and purifying the exhaled air of the confirmed cases or suspected cases.

The prior art related to the present invention is disclosed in Korean Utility Model Application Laid-Open No. 20-2016-0001206 entitled ‘Yellow sand mask’.

### SUMMARY OF THE INVENTION

Accordingly, the present invention has been made in view of the above-mentioned problems occurring in the prior art, and it is an object of the present invention to provide to an air purifying mask capable of filtering inhaled and exhaled air that filters and purifies, at the time when external air is absorbed to the interior thereof and even at the time when the exhaled air is exhausted therefrom, a variety of bacteria and fine foreign substances contained in the exhaled air, thereby in advance preventing the infection of the respiratory diseases caused by the respiration of a carrier or suspected patient.

It is another object of the present invention to provide to an air purifying mask capable of filtering inhaled and exhaled air that is configured to allow an intake part through which external air is introduced into the mask to be open at the time of air intake and to be closed at the time of air exhaust and also to completely prevent occurrence of a given gap on the intake part at the time of the air exhaust, thereby keeping the exhaled air of a carrier or suspected patient from leaking to the intake part.

It is yet another object of the present invention to provide to an air purifying mask capable of filtering inhaled and exhaled air that diffuses and exhausts the exhaled air collected to a given portion of a filtering part at the time of air exhaust, thereby effectively performing filtration and purification of the exhaled air.

To accomplish the above-mentioned objects, according to the present invention, there is provided an air purifying mask capable of filtering inhaled and exhaled air including: an inhaled air opening part open at the time of inhalation and closed at the time of exhalation in such a manner as to be coupled to an intake air filtering part for filtering and introducing external air; and an exhaled air opening part open at the time of the exhalation and closed at the time of the inhalation in such a manner as to be coupled to an exhaust air filtering part for filtering and exhausting the exhaled air.

According to the present invention, desirably, the intake air filtering part includes: at least one intake air filter for filtering the external air; and an intake air filter coupling body coupled to the intake air filter on one side thereof and connected to an intake air port formed on one side of the mask on the opposing side to one side thereof, and the exhaust air filtering part includes: at least one exhaust air filter for filtering the exhaled air; and an exhaust air filter coupling body coupled to the exhaust air filter on one side thereof and connected to an exhaust air port formed on the other side of the mask on the opposing side to one side thereof.

According to the present invention, desirably, the intake air filter includes: an intake air pre-filter for primarily filtering the external air; and an intake air HEPA filter for secondarily filtering the external air primarily filtered through the intake air pre-filter, and the exhaust air filter includes: an exhaust air pre-filter for primarily filtering the exhaled air; and an exhaust air HEPA filter for secondarily filtering the exhaled air primarily filtered through the exhaust air pre-filter.

According to the present invention, desirably, the inhaled air opening part includes: an intake air inlet body having at least one intake air hole adapted to pass the external air purified in the intake air filtering part therethrough; and an intake air plate film body for opening the intake air hole at the time of the inhalation and closing the intake air hole at the time of the exhalation, and the exhaled air opening part comprises: an exhaust air outlet body having at least one exhaust air hole adapted to pass the exhaled air therethrough toward the exhaust air filtering part; and an exhaust air plate film body for opening the exhaust air holes at the time of the exhalation and closing the exhaust air holes at the time of the inhalation.

According to the present invention, desirably, the intake air plate film body includes a plate film cap bent along the edge periphery thereof toward the intake air inlet part or formed stepped.

According to the present invention, desirably, the inhaled air opening part further includes: a plate film contacting material disposed along the edge periphery of the intake air inlet body or the intake air hole; or a plate film contacting layer formed disposed on a contacted portion of the intake air inlet body with the intake air plate film body.

According to the present invention, desirably, the intake air filter coupling body includes: a first inhaled air region having a diameter corresponding to a diameter of the intake air filter; and a second inhaled air region formed stepped to a smaller diameter than the first inhaled air region, and the exhaust air filter coupling body includes: a first exhaled air region in which the exhaled air exhausted from the exhaust air port flows to the exhaust air filter; and a second exhaled air region formed stepped to a larger diameter than the first exhaled air region.

According to the present invention, desirably, the intake air filtering part further includes the inhaled air opening part formed on any one of the first inhaled air region and the second inhaled air region and the exhaust air filtering part further comprises the exhaled air opening part formed on any one of the first exhaled air region and the second exhaled air region.

According to the present invention, desirably, the exhaust air filtering part includes a diffusion guide disposed on any one of the first exhaled air region and the second exhaled air region of the exhaust air filter coupling body to diffuse the exhaled air.

According to the present invention, desirably, the air purifying mask further includes a face contacting material formed on at least a portion contacting with a wearer's face.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG.1 is a front view showing an air purifying mask capable of filtering inhaled and exhaled air according to the present invention;

FIG.2 is a sectional view showing an inhaled air opening part and an intake air filtering part of FIG.1;

FIG.3 is a sectional view showing another example of an intake air plate film body of FIG.2;

FIG.4 is a sectional view showing another example of an exhaled air opening part and an exhaust air filtering part of FIG.1;

FIG.5 is a sectional view showing another example of an exhaust air plate film body of FIG.4;

FIG.6 is a schematic sectional view showing a plate film cap disposed on an intake air inlet part of FIGS.2 and 3;

FIG.7 is a schematic sectional view showing the exhaled air opening part of FIGS.2 and 3 having a plate film contacting material disposed thereon;

FIG.8 is a schematic sectional view showing the exhaled air opening part of FIGS.2 and 3 having a plate film contacting layer disposed thereon;

FIG.9 is a sectional view showing another example of the intake air filtering part of FIG.2;

FIG.10 is a sectional view showing another example of the exhaust air filtering part of FIG.4;

FIG.11 is a schematic sectional view showing the exhaust air filtering part of FIG.10 having a diffusion guide disposed thereon;

FIGS.12a to 12c are front views showing various examples of the diffusion guide of FIG.11; and

FIG.13 is a rear view showing the air purifying mask capable of filtering inhaled and exhaled air according to the present invention.

### DETAILED DESCRIPTION OF THE PREFERRED EMBODIMENTS

Hereinafter, an explanation on an air purifying mask capable of filtering inhaled and exhaled air according to the present invention will be in detail given with reference to the attached drawing.

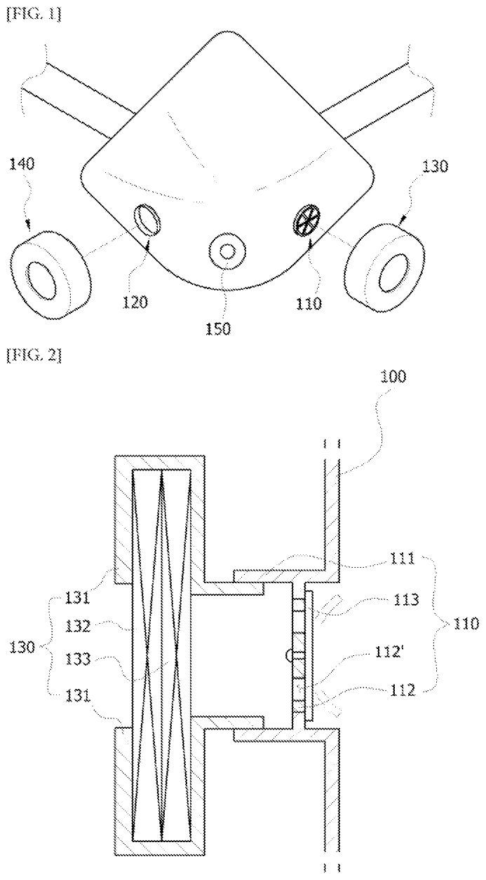

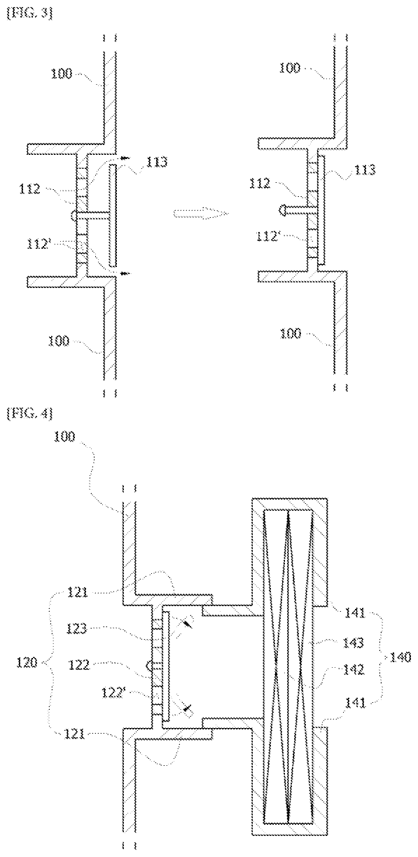

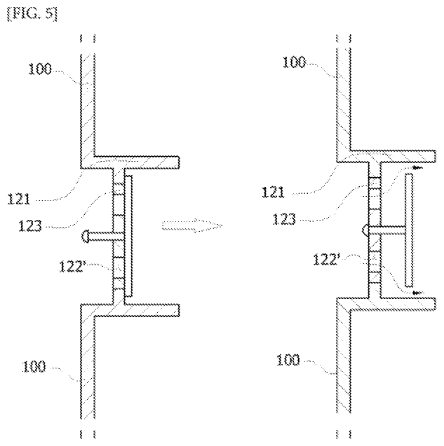

FIG.1 is a front view showing an air purifying mask capable of filtering inhaled and exhaled air according to the present invention, FIG.2 is a sectional view showing an inhaled air opening part and an intake air filtering part of FIG.1, FIG.3 is a sectional view showing another example of an intake air plate film body of FIG.2, FIG.4 is a sectional view showing another example of an exhaled air opening part and an exhaust air filtering part of FIG.1, and FIG.5 is a sectional view showing another example of an exhaust air plate film body of FIG.4.

As shown in FIG.1, an air purifying mask 100 capable of filtering inhaled and exhaled air according to the present invention includes an inhaled air opening part 110 and an exhaled air opening part 120.

The inhaled air opening part 110 is adapted to filter external air introduced into the mask 100 when a carrier or suspected patient (hereinafter, referred to as ‘wearer’) who wears the mask 100 inhales the external air and is thus configured to be open at the time when the wearer inhales the external air to perform intake of the air and to be closed at the time when he or she exhales the inhaled air. Further, the inhaled air opening part 110 is coupled to an intake air filtering part 130 for filtering the external air.

A detailed explanation on the inhaled air opening part 110 according to the present invention will be given later after the intake air filtering part 130 is described below.

According to the present invention, as shown in FIG.2, the intake air filtering part 130 includes an intake air filter coupling body 131 whose one side connected to an intake air port 111 formed on one side of the mask 100 to locate an intake air filter at the interior thereof. The intake air filter coupling body 131 and the intake air port 111 are screw-coupled to each other or formed unitarily with each other.

The intake air filter includes an intake air pre-filter 132 disposed on one side of the intake air filter coupling body 131 to primarily filter the external air and an intake air

HEPA filter 133 disposed between the intake air pre-filter 132 and the intake air port 111 to secondarily filter the external air, so that through the intake air pre-filter 132 and the intake air HEPA filter 133, the external air introduced into the intake air filtering part 130 is purified. In detail, the intake air pre-filter 132 and the intake air HEPA filter 133 are disposed on the intake air filter coupling body 131 in such a manner as to be located on a moving path of the air introduced upon the wearer's inhalation, thereby purifying the introduced air and removing the bacteria and fine foreign substances contained in the external air.

The intake air pre-filter 132 is made of an antibacterial material and is adapted to remove relatively large dust, fungi, hair, and animal hair.

The intake air HEPA filter 133 is made of a non-woven fabric on the front surface thereof and has excellent performance in removing fine dust, thereby performing a nano silver and catechin antibacterial/anti-fungal treatment and removing cigarette smoke, virus, bacteria, and so on.

Next, the inhaled air opening part 110 includes an intake air inlet body 112 having at least one or more intake air holes 112′ formed thereon to pass the external air purified through the intake air filtering part 130 therethrough toward the inside of the mask 100, upon the wearer's inhalation, and an intake air plate film body 113 for opening or closing the intake air holes 112′.

The intake air inlet body 112 has a shape of a plate on which the intake air holes 112′ are formed, as shown by the inhaled air opening part 110 in FIG.1, and if necessary, a filter for filtering the external air passing through the intake air holes 112′ may be disposed.

As shown in FIG.2, the intake air plate film body 113 is located on the inside of the intake air inlet body 112 with respect to the mask 100, and a portion of the intake air plate film body 113 is fixed to the intake air inlet body 112, so that the intake air plate film body 113 is bent toward the inside of the mask 100 with respect to the fixed portion thereof, upon the wearer's inhalation.

In this case, the intake air plate film body 113 is made of silicone or rubber having strong bonding properties, but it may be made of various materials only if it is bendable, without being limited thereto.

Accordingly, the wearer can introduce the external air into the mask 100 through the exhaled air opening part 120 as will be discussed later in the state of being closed when he or she inhales and through the intake air plate film body 113 bent by an intake air pressure to thus open the intake air holes 112′.

Normally, the intake air plate film body 113 is desirably kept brought into close contact with the intake air inlet body 112.

Unlike the intake air plate film body 113 bent in the state where a portion thereof is fixed to the intake air inlet body 112, on the other hand, the intake air plate film body 113 is coupled to the intake air inlet body 112 in such a manner as to move in an introduction direction of the external air, as shown in FIG.3.

In case of the intake air plate film body 113 having such a structure, in the same manner as above, the wearer can introduce the external air into the mask 100 through the exhaled air opening part 120 as will be discussed later in the state of being closed when he or she inhales and through the intake air plate film body 113 moving to the inside of the mask 100 by an intake air pressure to thus open the intake air holes 112′.

As shown in FIGS.1 and 4, the exhaled air opening part 120 is adapted to filter the exhaled air exhausted when the wearer exhales and is thus configured to be closed at the time when the wearer inhales to breathe and to be open at the time when he or she exhales to exhaust the inhaled air. Further, the exhaled air opening part 120 is coupled to an exhaust air filtering part 140 for filtering the exhaled air.

A detailed explanation on the exhaled air opening part 120 according to the present invention will be given later after the exhaust air filtering part 140 is described below.

According to the present invention, as shown in FIG.4, the exhaust air filtering part 140 includes an exhaust air filter coupling body 141 whose one side connected to an exhaust air port 121 formed on one side of the mask 100 to locate an exhaust air filter at the interior thereof. The exhaust air filter coupling body 141 and the exhaust air port 121 are screw-coupled to each other or formed unitarily with each other.

The exhaust air filter includes an exhaust air pre-filter 142 disposed on one side of the exhaust air filter coupling body 141 adjacent to the exhaust air port 121 to primarily filter the exhaled air and an exhaust air HEPA filter 143 disposed on the other side of the exhaust air pre-filter 142 to secondarily filter the exhaled air, so that through the exhaust air pre-filter 142 and the exhaust air HEPA filter 143, the exhaled air exhausted toward the exhaust air filtering part 140 is purified. In detail, the exhaust air pre-filter 142 and the exhaust air HEPA filter 143 are disposed on the exhaust air filter coupling body 141 in such a manner as to be located on a moving path of the exhaled air exhausted upon the wearer's exhalation, thereby purifying the exhaled air exhausted and removing the bacteria and fine foreign substances contained in the exhaled air of the wearer.

The exhaust air pre-filter 142 is made of an antibacterial material and is adapted to remove relatively large dust, fungi, hair, and animal hair.

The exhaust air HEPA filter 143 is made of a non-woven fabric on the front surface thereof and has excellent performance in removing fine dust, thereby performing a nano silver and catechin antibacterial/anti-fungal treatment and removing cigarette smoke, virus, bacteria, and so on.

Next, the exhaled air opening part 120 includes an exhaust air outlet body 122 having at least one exhaust air holes 122′ formed thereon to pass the exhaled air through the exhaust air filtering part 140, upon the wearer's exhalation, and an exhaust air plate film body 123 for opening or closing the exhaust air hole 122′.

Like the intake air inlet body 112 as mentioned above, the exhaust air outlet body 122 has a shape of a plate on which the exhaust air holes 122′ are formed, and if necessary, a filter for filtering the external air passing through the exhaust air holes 122′ may be disposed.

As shown in FIG.4, the exhaust air plate film body 123 is located on the side of the exhaust air outlet body 122 with respect to the exhaust air filtering part 140, and a portion of the exhaust air plate film body 123 is fixed to the exhaust air outlet body 122, so that the exhaust air plate film body 123 is bent toward the exhaust air filtering part 140 with respect to the fixed portion thereof, upon the wearer's exhalation.

In this case, the exhaust air plate film body 123 is made of silicone or rubber having strong bonding properties, but it may be made of various materials only if it is bendable, without being limited thereto.

Accordingly, the wearer can exhaust the exhaled air to the outside of the mask 100 through the inhaled air opening part 110 as mentioned above in the state of being closed at the time of the exhalation and through the exhaust air plate film body 123 bent by an exhaled air pressure to thus open the exhaust air holes 122′.

Normally, the exhaust air plate film body 123 is desirably kept brought into close contact with the exhaust air outlet body 122.

Unlike the exhaust air plate film body 123 bent in the state where a portion thereof is fixed to the exhaust air outlet body 122, on the other hand, the exhaust air plate film body 123 is coupled to the exhaust air outlet body 122 in such a manner as to move in an introduction direction of the external air, as shown in FIG.5.

In case of the exhaust air plate film body 123 having such a structure, in the same manner as above, the wearer exhausts the exhaled air to the outside of the mask 100 through the inhaled air opening part 110 as mentioned above in the state of being closed at the time of exhalation and through the exhaust air plate film body 123 moving to the exhaust air filtering part 140 by the exhaled air pressure to thus open the exhaust air holes 122′.

As shown in FIG.1, further, the air purifying mask 100 according to the present invention includes an auxiliary intake part 150 disposed on the center between the inhaled air opening part 110 and the exhaled air opening part 120.

The auxiliary intake part 150 is connected to an oxygen tank in an ambulance, and if the oxygen tank is connected to the mask 100, the purified air in the oxygen tank is supplied through the auxiliary intake part 150 to the interior of the mask 100, so that the wearer, especially, the weak patient who is hard to breathe due to his or her weak intake force, like an emergency patient, can breathe more conveniently and easily.

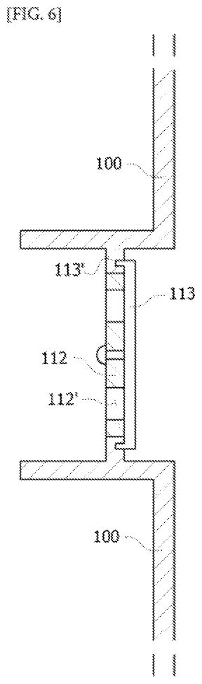

FIG.6 is a schematic sectional view showing a plate film cap disposed on the intake air inlet part of FIGS. 2 and 3.

As shown in FIG.6, the intake air plate film body 113 of the air purifying mask 100 according to the present invention includes a plate film cap 113′ formed to in advance prevent a gap between the intake air inlet body 112 and the intake air plate film body 113 from occurring normally or upon air exhaustion.

The plate film cap 113′ of the intake air plate film body 113 is formed by bending the edge periphery of the intake air plate film body 113 toward the intake air inlet body 112, as shown in FIG.6.

In this case, the mask 100 or the intake air inlet body 112 desirably has a plate film cap groove formed correspondingly to the plate film cap 113′ to insert the plate film cap 113′ thereinto.

Through the plate film cap 113′ of the intake air plate film body 113, like this, the occurrence of the gap between the intake air inlet body 112 and the intake air plate film body 113 can be prevented more efficiently.

On the other hand, the plate film cap 113′ may be formed stepped between the edge periphery of the intake air plate film body 113 and the center thereof, while being not bent along the edge periphery of the intake air plate film body 113.

If necessary, of course, the plate film cap may be formed on the exhaust air plate film body 123.

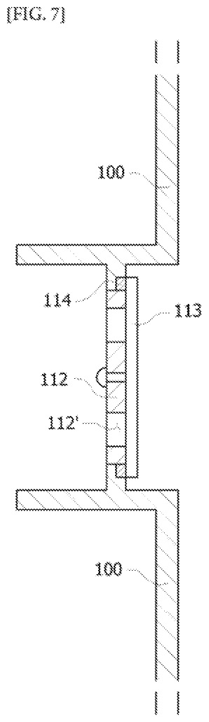

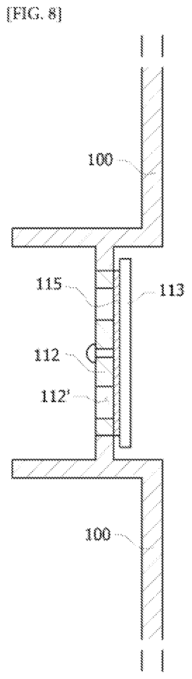

FIG.7 is a schematic sectional view showing the exhaled air opening part of FIGS.2 and 3 having a plate film contacting material disposed thereon and FIG.8 is a schematic sectional view showing the exhaled air opening part of FIGS.2 and 3 having a plate film contacting layer disposed thereon.

As shown in FIG.7, the inhaled air opening part 110 of the air purifying mask 100 according to the present invention includes a plate film contacting material 114 adapted to in advance prevent a gap between the intake air plate film body 113 and the intake air inlet body 112 from occurring normally or upon air exhaustion.

The plate film contacting material 114 is desirably disposed along the edge periphery of the intake air inlet body 112 or the intake air hole 112′. The plate film contacting material 114 is made of silicone or rubber to improve the contacting force with the intake air plate film body 113, but it may be made of various materials only if it improves the contacting force with the intake air plate film body 113, without being limited thereto.

As shown in FIG.8, further, the inhaled air opening part 110 of the air purifying mask 100 according to the present invention includes a plate film contacting layer 115 adapted to in advance prevent a gap between the intake air plate film body 113 and the intake air inlet body 112 from occurring normally or upon air exhaustion.

The plate film contacting layer 115 is disposed between the intake air inlet body 112 and the intake air plate film body 113, as shown in FIG.8, and desirably has the corresponding area to the area of the intake air inlet body 112.

The plate film contacting layer 115 is made of silicone or rubber to improve the contacting force with the intake air plate film body 113, but it may be made of various materials only if it improves the contacting force with the intake air plate film body 113, without being limited thereto.

Like this, if the inhaled air opening part 110 is provided with the plate film contacting material 114 or the plate film contacting layer 115, the contacting force with the intake air plate film body 113 is improved to more effectively prevent the occurrence of the gap between the intake air plate film body 113 and the intake air inlet body 112 or the intake air hole 112′.

If necessary, of course, the plate film contacting material 114 or the plate film contacting layer 115 may be formed on the exhaled air opening part 120.

Now, an explanation on another example of each of the intake air filtering part 130 and the exhaust air filtering part 140 according to the present invention will be given.

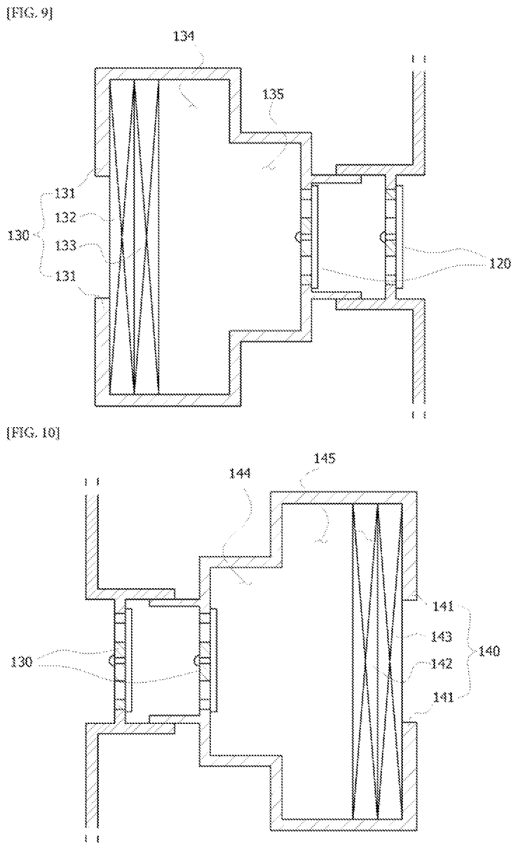

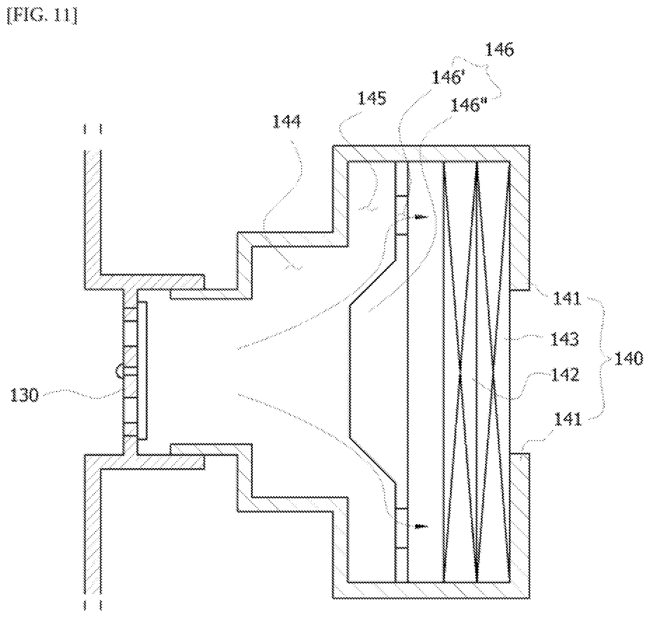

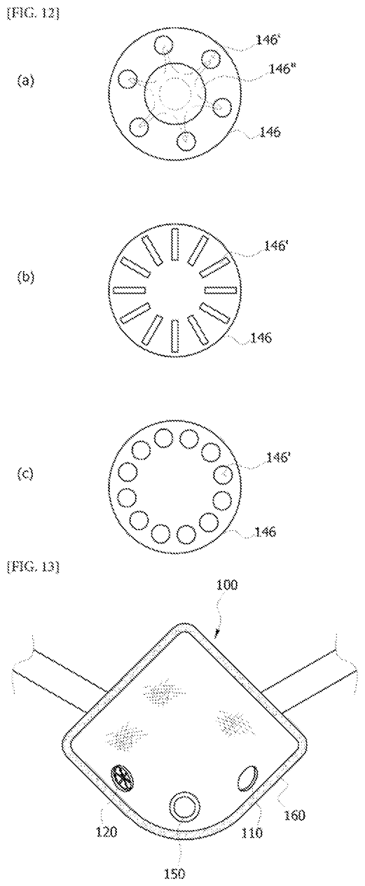

FIG.9 is a sectional view showing another example of the intake air filtering part of FIG.2, FIG.10 is a sectional view showing another example of the exhaust air filtering part 140 of FIG.4, FIG.11 is a schematic sectional view showing the exhaust air filtering part of FIG.10 having a diffusion guide disposed thereon, and FIGS.12a to 12c are front views showing various examples of the diffusion guide of FIG.11.

As shown in FIG.9, the intake air filter coupling body 131 of the intake air filtering part 130 includes a first inhaled air region 134 in which the external air passed and filtered through the intake air filter flows and a second inhaled air region 135 extended from the first inhaled air region 134 and having a smaller volume than the first inhaled air region 134.

The first inhaled air region 134 of the intake air filter coupling body 131 has the corresponding diameter to the intake air filter, and the second inhaled air region 135 is stepped smaller than a diameter of the first inhaled air region 134. A diameter of the first inhaled air region 134 is larger than a diameter of the intake air port 111.

Such a configuration prevents the external air in the interior of the intake air filter coupling body 131 having the larger diameter than the intake air port 111 from remaining in the intake air port 111 due to the failure in the introduction into the intake air port 111. In detail, the diameters of the first inhaled air region 134, the second inhaled air region 135 and the intake air port 111 become small in the order mentioned, thereby more efficiently performing the intake of the external air.

As shown in FIG.10, the exhaust air filter coupling body 141 of the exhaust air filtering part 140 includes a first exhaled air region 144 in which the exhaled air exhausted from the exhaust air port 121 flows and a second exhaled air region 145 extended from the first exhaled air region 144 and having a larger volume than the first exhaled air region 144.

The second exhaled air region 145 of the exhaust air filter coupling body 141 has the corresponding diameter to the exhaust air filter, and the first exhaled air region 144 is stepped smaller than a diameter of the second exhaled air region 144.

This is aimed to diffuse the exhaled air exhausted from the exhaust air port 121, and the exhaled air flowing from the first exhaled air region 144 to the second exhaled air region 145 passes through the front surface of the exhaust air filter and is thus exhausted, thereby more effectively performing the filtration and purification of the exhaust air filter.

So as to prevent the inhaled air from being exhausted normally or upon the intake of air, the exhaled air opening part 120 may be disposed further on the first exhaled air region 144 or the second exhaled air region 145.

On the other hand, as shown in FIGS.11 and 12a, the exhaust air filtering part 140 includes a diffusion guide 146 disposed on any one of the first exhaled air region 144 and the second exhaled air region 145.

The diffusion guide 146 includes at least one diffusion hole 146′ formed along the edge periphery thereof to allow the exhaled air to be diffused to the inner peripheral wall of the exhaust air filter coupling body 141 and a protruding diffusion portion 146″ formed on the center thereof to allow a portion thereof to circularly protrude therefrom.

According to the present invention, the diffusion guide 146 has the protruding diffusion portion 146″, but as shown in FIGS. 12b and 12c, the diffusion guide 146 may be freely formed, without having any protruding diffusion portion 146″.

Through the diffusion guide 146, like this, the exhaled air is induced to the inner peripheral walls of the first exhaled air region 144 and the second exhaled air region 145 and diffusedly flows to the exhaust air filter, as mentioned above.

FIG.13 is a rear view showing the air purifying mask capable of filtering inhaled and exhaled air according to the present invention.

The air purifying mask 100 capable of filtering the inhaled and exhaled air according to the present invention has a face contacting material 160 formed on at least a portion of the inside contacting with the wearer's face.

The face contacting material 160, which comes into close contact with the wearer's face, is desirably made of a material having a good contacting force like silicone.

As described above, the air purifying mask capable of filtering the inhaled and exhaled air according to the present invention can filter and purify, at the time when the external air is absorbed to the interior thereof and even at the time when the exhaled air is exhausted therefrom, a variety of bacteria and fine foreign substances contained in the exhaled air.

Accordingly, the air purifying mask according to the present invention can in advance prevent the infection of the respiratory diseases caused by the respiration of a carrier or suspected patient.

In addition, the air purifying mask according to the present invention can be configured to allow the intake part through which the external air is introduced into the mask to be open at the time of air intake and to be closed at the time of air exhaust and also to completely prevent the occurrence of a given gap on the intake part at the time of the air exhaust.

Accordingly, the air purifying mask according to the present invention can keep the exhaled air of a carrier or suspected patient from leaking to the intake part in which the exhaled air cannot be filtered.

Further, the air purifying mask according to the present invention can diffuse and exhaust the exhaled air collected to a given portion of the filtering part at the time of air exhaust, thereby effectively performing filtration and purification of the exhaled air.

The air purifying mask according to the present invention can be applied to a field of disease control and prevention, especially, a field of a mask and a gas mask, and products manufactured in the corresponding fields thereto or in the similar or related fields thereof, thereby improving the reliability and competitiveness for the products in the corresponding fields.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

1. An air purifying mask capable of filtering inhaled and exhaled air, comprising:
an inhaled air opening part
an intake air filtering part,
wherein the inhaled air opening part opens at the time of inhalation and closes at the time of exhalation in such a manner as to be coupled to the intake air filtering part for filtering and introducing external air;
an exhaled air opening part; and
an exhaust air filtering part,
wherein the exhaled air opening part opens at the time of the exhalation and closesd at the time of the inhalation in such a manner as to be coupled to the exhaust air filtering part for filtering and exhausting an exhaled air.

2. The air purifying mask according to claim 1, wherein the intake air filtering part comprises:
at least one intake air filter for filtering the external air; and
an intake air filter coupling body coupled to the intake air filter on one side thereof and connected to an intake air port formed on one side of the mask on the opposing side to one side thereof, and
the exhaust air filtering part comprises:
at least one exhaust air filter for filtering the exhaled air; and
an exhaust air filter coupling body coupled to the exhaust air filter on one side thereof and connected to an exhaust air port formed on the other side of the mask on the opposing side to one side thereof.

3. The air purifying mask according to claim 2, wherein the intake air filter comprises:
an intake air pre-filter for primarily filtering the external air; and
an intake air HEPA filter for secondarily filtering the external air primarily filtered through the intake air pre-filter,
and the exhaust air filter comprises:
an exhaust air pre-filter for primarily filtering the exhaled air; and
an exhaust air HEPA filter for secondarily filtering the exhaled air primarily filtered through the exhaust air pre-filter.

4. The air purifying mask according to claim 1, wherein the inhaled air opening part comprises:
an intake air inlet body having at least one intake air hole adapted to pass the external air purified in the intake air filtering part therethrough; and
an intake air plate film body for opening the intake air hole at the time of the inhalation and closing the intake air hole at the time of the exhalation, and
the exhaled air opening part comprises:
an exhaust air outlet body having at least one exhaust air hole adapted to pass the exhaled air therethrough toward the exhaust air filtering part; and
an exhaust air plate film body for opening the exhaust air holes at the time of the exhalation and closing the exhaust air holes at the time of the inhalation.

5. The air purifying mask according to claim 4, wherein the intake air plate film body comprises a plate film cap bent along the edge periphery thereof toward the intake air inlet part or formed stepped.

6. The air purifying mask according to claim 4, wherein the inhaled air opening part further comprises: a plate film contacting material disposed along the edge periphery of the intake air inlet body or the intake air hole; or a plate film contacting layer formed disposed on a contacted portion of the intake air inlet body with the intake air plate film body.

7. The air purifying mask according to claim 4, wherein the intake air filter coupling body comprises:
a first inhaled air region having a diameter corresponding to a diameter of the intake air filter; and
a second inhaled air region formed stepped to a smaller diameter than the first inhaled air region, and
the exhaust air filter coupling body comprises:
a first exhaled air region in which the exhaled air exhausted from the exhaust air port flows to the exhaust air filter; and
a second exhaled air region formed stepped to a larger diameter than the first exhaled air region.

8. The air purifying mask according to claim 7, wherein the intake air filtering part further comprises the inhaled air opening part formed on any one of the first inhaled air region and the second inhaled air region and the exhaust air filtering part further comprises the exhaled air opening part formed on any one of the first exhaled air region and the second exhaled air region.

9. The air purifying mask according to claim 7, wherein the exhaust air filtering part comprises a diffusion guide disposed on any one of the first exhaled air region and the second exhaled air region of the exhaust air filter coupling body to diffuse the exhaled air.

10. The air purifying mask according to claim 1, further comprising a face contacting material formed on at least a portion contacting with a wearer's face.
